(19) United States
(12) Patent Application Publication
(10) Pub. No.: US 2015/0202187 A1
(43) Pub. Date: Jul. 23, 2015

(54) COMPOSITIONS AND METHODS FOR TREATMENT OF VITILIGO

(71) Applicants: Sarah BACUS, Hinsdale, IL (US); Diarmuid MORAN, (US)

(72) Inventors: Sarah Bacus, Hinsdale, IL (US); Diarmuid Moran, Chicago, IL (US)

(21) Appl. No.: 14/414,874

(22) PCT Filed: Jun. 26, 2013
(86) PCT No.: PCT/US13/47930

§ 371 Date: Jan. 14, 2015

Related U.S. Application Data
(60) Provisional application No. 61/664,481, filed on Jun. 26, 2012.

(51) Int. Cl. A61K 31/436; A61K 31/352; A61K 38/34; A61K 38/44; A61K 31/355; A61K 38/18; A61K 31/198

(52) U.S. Cl. CPC A61K 31/436; A61K 38/1825; A61K 31/352; A61K 31/198; A61K 38/44; A61K 31/355; A61K 38/34; C12Y 111/01009; C12Y 111/01006

(57) ABSTRACT

Compositions and methods are disclosed for treating hypomelanotic conditions such as vitiligo and promoting melanogenesis.

### TECHNICAL FIELD

The present invention relates to a novel skin composition that reduces the progression of hypomelanotic conditions such as vitiligo. The invention further relates to a composition containing a compound functioning to inhibit T-cells from attacking melanocytes. More specifically, the invention relates to a composition containing rapamycin as an active ingredient. In addition, the invention can contain growth factor (e.g. fibroblast growth factor, stem cell factor, endothelins), melanogenesis activators (e.g. alpha melanocyte stimulating hormone, L-Tyrosine/N-acetyl L-Tyrosine, L-DOPA, cAMP activators—forskolin, colforsin), and antioxidants (e.g. catalase, vitamin E) as additional active ingredients. The invention further relates to a composition for promoting the formation of collagen in the skin, wherein the composition comprises the aforementioned compound or compounds.

### BACKGROUND

Vitiligo is an autoimmune disease presenting with progressive loss of skin pigmentation. Vitiligo is a cutaneous disease in which melanocytes are destroyed in discrete patches, resulting in lightened areas of variable size and location distributed throughout the skin of the body. Melanocytes are cells located in the stratum basale (bottom layer) of the skin's epidermis. They are also located in the eye, ear, meninges, bones and heart. Melanocytes produce a pigment called melanin, a derivative of the amino acid tyrosine, through the process of melanogenesis. Melanogenesis is primarily regulated by α-melanocyte-stimulating hormone that binds to the Melanocortin-1 receptor (MC1R) on melanocytes leading to adenylate cyclase activation, elevation of the intracellular cyclic adenosine monophosphate (cAMP) content, and activation of protein kinase A (PKA). Increased PKA activity leads to increased tyrosinase activity (the enzyme responsible for the initiation of melanogenesis), dendrite formation and proliferation in melanocytes. In vitro, the melanogenic effects of α-melanocyte-stimulating hormone can be mimicked by pharmacologic agents that activate cAMP levels in melanocytes. Two major types of melanin are produced within the melanosomes of human melanocytes—eumelanin (brown/black pigments) and pheomelanin (yellow/red pigments). The type of melanin produced depends on the enzyme profile of melanocytes and the prevalent metabolism of the cells. Melanogenesis begins with the hydroxylation of the amino acid L-Tyrosine to form L-DOPA, which is catalyzed by the enzyme Tyrosinase. In the next step of the cascade, Tyrosinase rapidly oxidizes L-DOPA to form Dopaquinone which initiates Eu- or Pheo-melanogenesis. Once L-DOPA is formed, the further steps of melanogenesis occur spontaneously. Variations in the activity of melanocytes and the production of melanin is a primary determinant of human skin color. Vitiligo is a condition that causes skin depigmentation due to loss of function and/or death of melanocytes (pigment cells) in the epidermis producing milky-white patches on affected skin. The condition of vitiligo can also affect eye pigmentation and ear function, as melanin is expressed in both the ear and the uveal tract of the eye. The lightened lesions of the skin generally have greater susceptibility to the damaging effects of the sun, premature aging and possibly skin cancer. The disease occurs in up to 1% of the world population, generally during teenage years. The exact etiology of vitiligo is unknown but the most widely accepted view is that it is an autoimmune disease involving immune attack of melanocytes by both T and B cell dependent mechanisms. The progressive loss of melanocytes from depigmenting vitiliginous skin is typically associated with cellular infiltrates containing T lymphocytes. Infiltrating cytotoxic T cells with high affinity T cell receptors have likely escaped clonal deletion in the thymus, allowing such T cells to enter the circulation. It is thought that through the expression of cutaneous lymphocyte antigen, these T cells home to the skin where they express type 1-cytokines and mediate melanocyte apoptosis via the granzyme/perforin pathway. As this condition affects the skin and is readily visible to the public eye, there are many psychological and social problems that can result. Vitiligo can be cosmetically disfiguring and is a stigmatizing condition, often leading to psychological problems in daily life. Hence, there is a great need for continuing development of treatments that can be used to minimize the visible consequences of a condition such as vitiligo, as well as other conditions which manifest themselves as discoloration of the skin (aging spots, liver spots, etc.).

### SUMMARY OF THE INVENTION

Compositions are disclosed for cosmeceuticals that aid in the retardation of the progression of vitiligo. Methods for preparing cosmeceutical compositions for treating vitiligo are also disclosed. More specifically, the methods herein disclose the use of rapamycin for slowing and/or reversing the progression of hypomelanotic conditions such as vitiligo and the use of rapamycin and fibroblast growth factor and melanogenesis activators for sustaining the growth and survival of melanocytes, promoting collagen formation, and stimulating repigmentation. More specifically, the methods herein disclose the use of rapamycin and fibroblast growth factor for stimulating the survival, proliferation and differentiation of melanocytes. Methods also disclose the use of other agents in combination with rapamycin that function to increase melanogenesis in melanocytes including L-DOPA, L-phenylalanine, L-TYROSINE, N-ACETYL L-TYROSINE, FORSKOLIN, COLFORSIN (NKH477) and alpha melanocyte stimulating hormone. Methods also disclose the use of antioxidants such as vitamin E, glutathione peroxidase and Catalase in combination with rapamycin that function to sustain the survival of melanocytes against oxidative attack/stress which is commonly associated with melanocyte damage/death in vitiliginous lesions.

In one aspect of the invention, the composition contains a cosmeceutically effective amount of rapamycin and growth factor (e.g. fibroblast growth factor, stem cell factor, endothelins) in a cosmeceutically acceptable vehicle.

In certain aspects, the composition contains at least one cAMP activator (e.g. forskolin or colforsin).

In additional aspects, the composition contains at least one melanogenesis activator (e.g. alpha melanocyte stimulating hormone, L-DOPA, L-Tyrosine, N-Acetyl L-Tyrosine, L-phenylalanine).

In another aspect, the composition contains an antioxidant (e.g. vitamin E or catalase).

In yet another aspect of the invention, the composition contains vitamin E, glutathione peroxidase, catalase or other acceptable antioxidants to act as an antioxidants that may suppress the detrimental effects of oxidative stress on melanocytes that is commonly observed in vitiligo.

In another aspect, the composition contains a preservative.

In additional aspects of the invention, the composition contains tromethamine to act as cosmeceutically acceptable buffer.

In one aspect, the composition acts to promote the proliferation of melanocytes.

In another aspect, the composition acts to reduce the T-cell induced destruction of melanocytes.

In another aspect, the composition is a topical composition.

In yet another aspect, the composition is a leave-on product.

In another aspect, the composition contains from about 0.001 to 2 weight percent rapamycin, or from about 0.1 to 0.5 weight percent rapamycin, or about 0.15 weight percent rapamycin.

In yet another aspect, the composition contains from about 0.0000001 to 0.1 weight percent growth factor, or from about 0.000001 to 0.00001 weight percent growth factor, or about 0.000005 weight percent growth factor.

In an alternate aspect, the composition contains from about 0.001 to 2 weight percent cAMP activator, or from about 0.05 to 0.5 weight percent cAMP activator, or about 0.1 weight percent cAMP activator.

In an additional aspect, the composition contains from about 0.001 to 0.1 weight percent alpha melanocyte stimulating hormone, or about 0.005 to 0.05 weight percent alpha melanocyte stimulating hormone, or about 0.01 weight percent alpha melanocyte stimulating hormone.

In another aspect, the composition contains from about 0.01 to 5 weight percent L-Tyrosine or N-Acetyl L-Tyrosine, or from about 1 to 3 weight percent L-Tyrosine or N-Acetyl L-Tyrosine, or about 2 weight percent L-Tyrosine or N-Acetyl L-Tyrosine.

In yet another aspect, the composition contains about 0.001 to 1 weight percent L-DOPA, or about 0.05 to 0.5 weight percent L-DOPA, or about 0.3 weight percent L-DOPA.

In an alternate aspect, the composition contains about 1 to 10 weight percent Vitamin E.

In yet another aspect, the composition contains about 0.00001 to 0.1 weight percent catalase.

In one aspect, the composition contains a cosmeceutically effective amount of rapamycin, growth factor, cAMP activator, alpha melanocyte stimulating hormone, melanogenesis inhibitor, antioxidants, and preservative in a cosmeceutically acceptable medium.

In another aspect, the composition containing a cosmeceutically effective amount of rapamycin, growth factor, cAMP activator, alpha melanocyte stimulating hormone, melanogenesis inhibitor, antioxidants, and preservative in a cosmeceutically acceptable medium is topically administered.

In yet another aspect, a composition of the invention treats vitiligo.

In yet another aspect, a composition of the invention reduces the T-cell induced destruction of melanocytes.

In another aspect, the T cells that target melanocytes are inhibited by topical administration of a composition of the invention.

In one aspect, a composition of the present invention contains from about 0.15% to 2% rapamycin, about 0.1% to 2% cAMP activator (e.g. forskolin or colforsin), about 1% to 5% melanogenesis activator (e.g. L-Tyrosine or N-Acetyl L-Tyrosine), and about 5% to 10% antioxidant (e.g. vitamin E).

### DESCRIPTION OF THE DRAWINGS

These and other advantages of the present invention will be readily understood with reference to the following specifications and attached drawings wherein:

FIG. 1. is Western blot of protein expression in HL60 cells treated with increasing doses of rapamycin (0.1 μM to 100 μM) for 24 hours.

FIG. 2 Depicts the proliferation of untreated and rapamycin-treated human melanocytes.

FIG. 3 Depicts the proliferation of untreated and FGF treated human melanocytes.

FIG. 4 Depicts the effect of various agents of the present invention on melanogenesis in human melanocytes in vitro.

FIG. 5 Depicts the increased melanogenesis in primary human melanocytes treated with active ingredients of the present invention.

FIG. 6 Depicts the increased proliferation of primary human melanocytes when treated with active ingredients of the present invention.

FIG. 7 Depicts a Western blot protein expression of survival signals in primary human melanocytes treated with active ingredients of the present invention.

FIG. 8 a-b Depict topical treatment of a subject with a composition of the present invention.

FIG. 9 Depicts topical treatment of a subject with a composition of the present invention.

FIG. 10 Depicts increased melanogenesis in cultured primary melanocytes treated with L-Tyrosine, Alpha-MSH, Forskolin, Rapamycin or combinations thereof for 96 hours.

### DETAILED DESCRIPTION

Embodiments of the present invention will be described herein below with reference to the accompanying drawings. In the following description, well-known functions or constructions are not described in detail because they may obscure the invention in unnecessary detail. Described herein are a number of compositions, methods, and formulations that may be applied to greatly improve the appearance of vitiligo afflicted skin. The term “a” in the present invention can mean more than one, is singular, plural or both. Specifically, the following embodiments are directed to compositions and methods using rapamycin.

In one aspect, the present invention is based on the discovery that compounds, such as rapamycin—an mTOR inhibitor, actively inhibit T-cells and inhibit the process of T-cell maturation that target melanocytes in vitiliginous skin and increase melanocytes. It has been further discovered that the use of rapamycin in cosmeceutically acceptable vehicle is an effective treatment for vitiligo when administered topically. Accordingly, one embodiment of the present invention is directed to a composition comprising a cosmeceutically acceptable vehicle and rapamycin.

As used herein, cosmeceutically acceptable and cosmeceutically effective refer to compositions and methods that bring about the desired result. Desired results may include inhibiting T-cells or inhibiting the process of T-cell maturation in melanocytes, providing repigmentation of the skin, increasing melanocyte production, or a combination of these effects. Cosmeceutical compositions and methods may include, but are not limited to, for example use as cosmetics, drugs, therapeutics, pharmaceuticals, biologic, cure, essence, medicament, medication, medicinal, medicine, pill, potion, prescription, remedy, tonic, any of the above which could be FDA-approved.

The response of vitiligo afflicted skin cells to treatment with rapamycin over a course of treatment (e.g., twice a day application of 2.5 uM cosmeceutical rapamycin composition) has shown response via reduction of the discoloration and reduction in the progression of the disease. In another aspect of the present invention, the use of rapamycin and fibroblast growth factor (FGF) stimulates the survival, proliferation and differentiation of melanocytes.

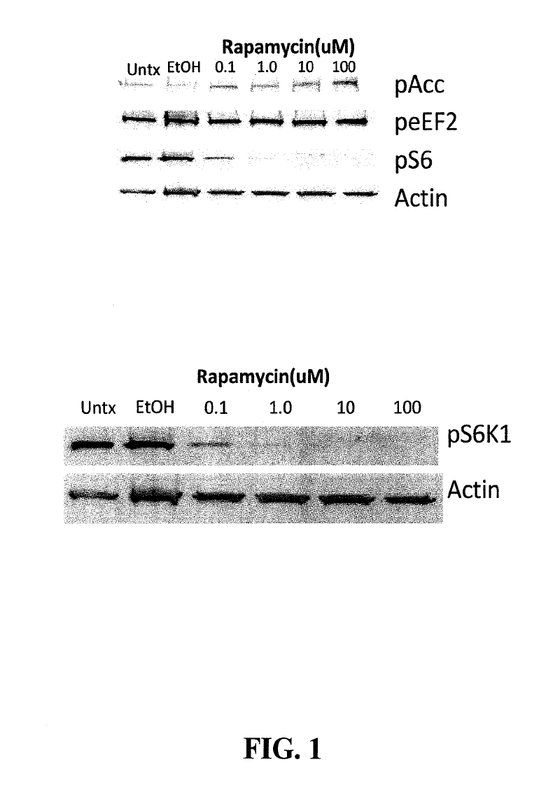

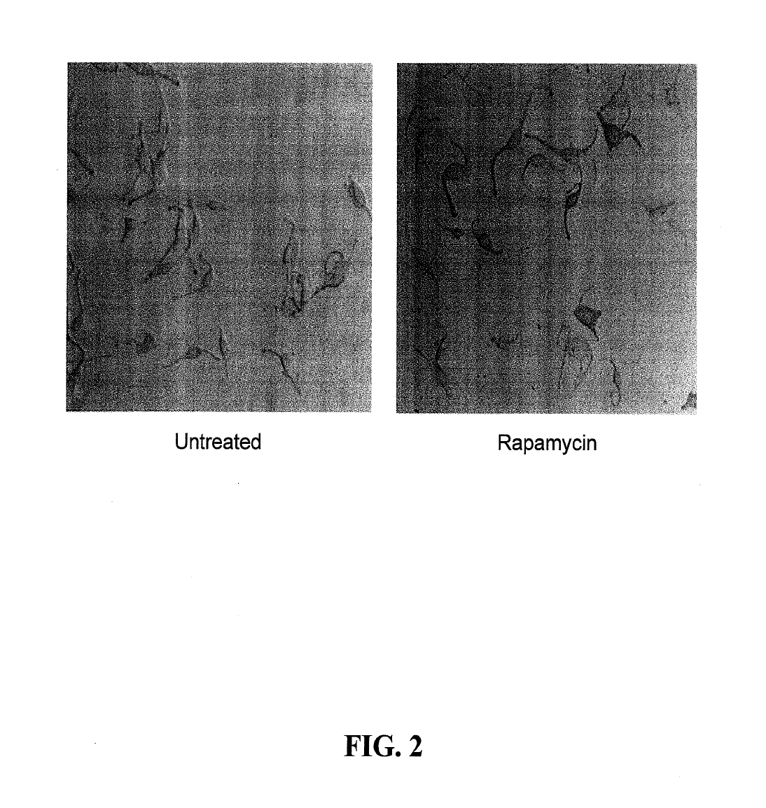

Rapamycin (also called sirolimus) inhibits immune attack by inhibiting the response to interleukin-2 (IL-2) and blocking activation of T- and B-cells. Rapamycin binds to the cytosolic protein FK-binding protein 12 (FKBP12) and inhibits the mammalian target of rapamycin (mTOR) pathway by directly binding the mTOR Complex 1 (mTORC1). Additionally, the present inventors have shown that topical administration of rapamycin promotes melanogenesis in cultured melanocytes and in the skin, thereby increasing skin pigmentation. For example, FIG. 1 depicts a western blot of lysates from HL60 cells, a model system for studying human myeloid cell differentiation, which were treated with rapamycin for 24 hours. As can be seen in FIG. 1, treatment with rapamycin decreased expression of phospho S6K1, a downstream substrate of mTOR. Additionally, FIG. 2 depicts human melanocytes that were treated with rapamycin (0.01 μM) for 96 hours. As compared to untreated melanocytes, rapamycin-treated melanocytes demonstrate increased melanogenesis.

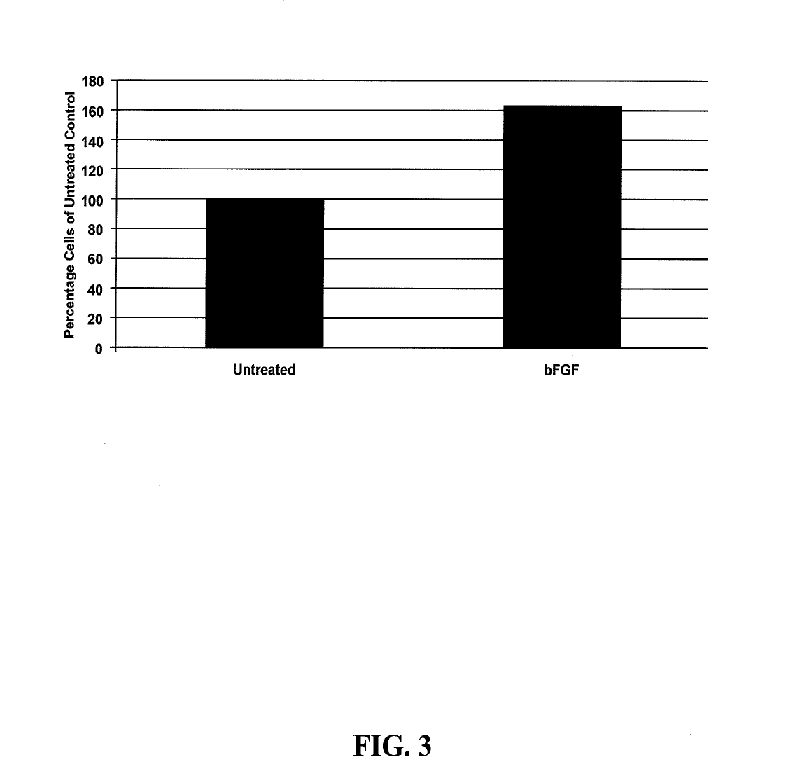

In some embodiments, the present invention is directed to a composition comprising a cosmeceutically acceptable carrier, rapamycin and growth factors such as fibroblast growth factor (FGF), Stem cell Factor (SCF) or Endothelins. As depicted in FIG. 3, bFGF increases proliferation of primary human melanocytes. In FIG. 3, treated Primary Human melanocytes were treated with bFGF (10 ng/mL) with DMSO as untreated control for 96 h. Growth Factors such as basic Fibroblast Growth Factor, Stem Cell Factor and Endothelins are important regulators of melanocyte differentiation, migration, proliferation and survival. Growth factors can therefore protect cells from destruction induced by immune cells. Furthermore, growth factors such as those described, can potentially increase migration of melanoblasts (melanocyte precursor cells) from peripheral regions of vitiligo patches or from hair follicles into the vitiligo lesion thus allowing repigmentation.

In other embodiments, the present invention is directed to a composition comprising rapamycin, a cosmeceutically acceptable carrier and antioxidants such as Vitamin E, Glutathione peroxidase, Catalase or combinations thereof.

Patients with vitiligo often have low catalase levels and/or high H2O2 in their involved and uninvolved epidermis which is inhibitory to melanogenesis. Antioxidants function to suppress oxidative stress in the melanocyte environment thus protecting melanocytes from oxidative stress induced death and/or loss of melanogenic function.

In other embodiments, the present invention is directed to a composition comprising a cosmeceutically acceptable vehicle and melanogenesis substrates such as l-DOPA, L-Tyrosine (or N-acetyl L-Tyrosine), L-phenylalanine, or combinations thereof.

In other embodiments, the present invention is directed to a composition comprising rapamycin, a cosmeceutically acceptable carrier and melanogenesis stimulators such as alpha-melanocyte stimulating hormone (alpha-MSH) or cAMP activators such as forskolin, colforsin or combinations thereof.

Melanogenesis is primarily regulated by α-melanocyte-stimulating hormone that binds to the Melanocortin-1 receptor (MC1R) on melanocytes leading to adenylate cyclase activation, elevation of the intracellular cyclic adenosine monophosphate (cAMP) content, and activation of protein kinase A (PKA). Increased PKA activity leads to increased tyrosinase expression and activity (the key enzyme that catalyzes the conversion of L-Tyrosine to L-DOPA, thus initiating melanogenesis), dendrite formation and proliferation in melanocytes. The melanogenic effects of α-melanocyte-stimulating hormone can be mimicked by pharmacologic agents that activate cAMP such as forskolin, or the water soluble derivative colforsin (NKH477) which lead to direct activation of melanogenesis in melanocytes. Two major types of melanin are produced within the melanosomes of human melanocytes—eumelanin (brown/black pigments) and pheomelanin (yellow/red pigments). The type of melanin produced depends on the enzyme profile of melanocytes and the prevalent metabolism. The biochemical pathways of melanogenesis begins with the hydroxylation of the amino acid L-Tyrosine to form L-DOPA, which is catalyzed by the enzyme, Tyrosinase. In the next step of the cascade, Tyrosinase rapidly oxidizes L-DOPA to form Dopaquinone which initiates Eu- or Pheo-melanogenesis. Once L-DOPA is formed, the further steps of melanogenesis occur spontaneously. Supplementation of melanocytes with melanogenesis substrates such as L-DOPA, L-Tyrosine (or water soluble derivative—N-Acetyl L-tyrosine) or L-phenylalanine (an L-Tyrosine precursor) stimulates melanogenesis. Thus supplementation of melanocytes with alpha-MSH, forskolin, colforsin, L-DOPA, L-Tyrosine, N-Acetyl L-Tyrosine, L-phenylalanine or combinations thereof can enhance melanogenesis in human melanocytes and thus increase skin pigmentation in hypomelanotic conditions such as vitiligo.

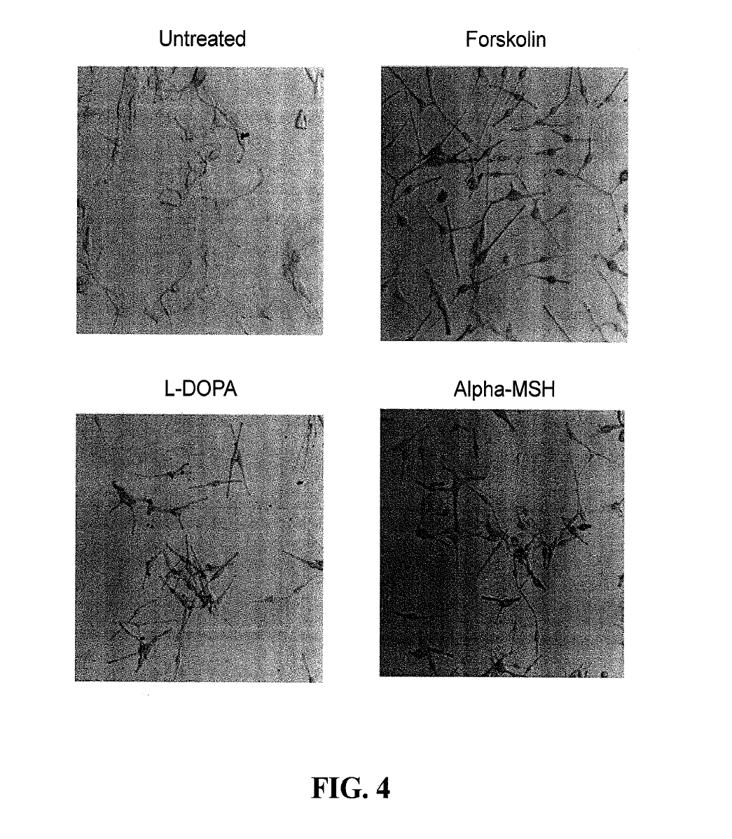

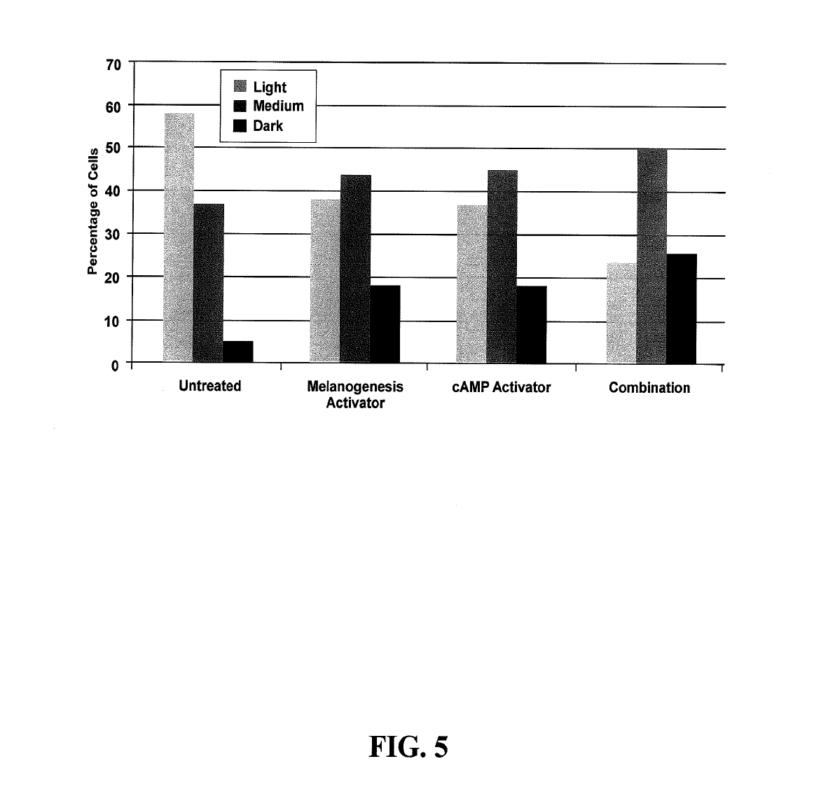

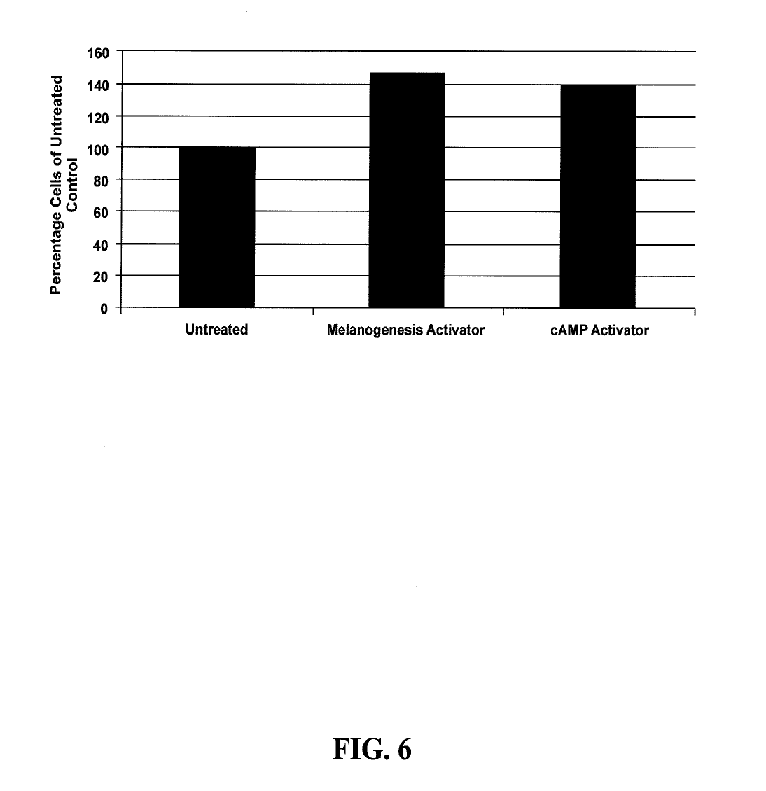

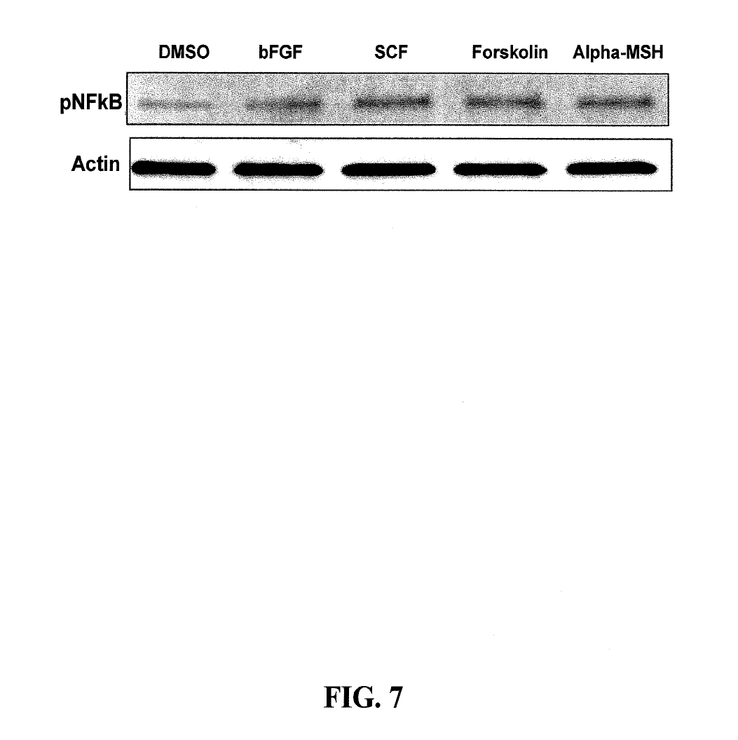

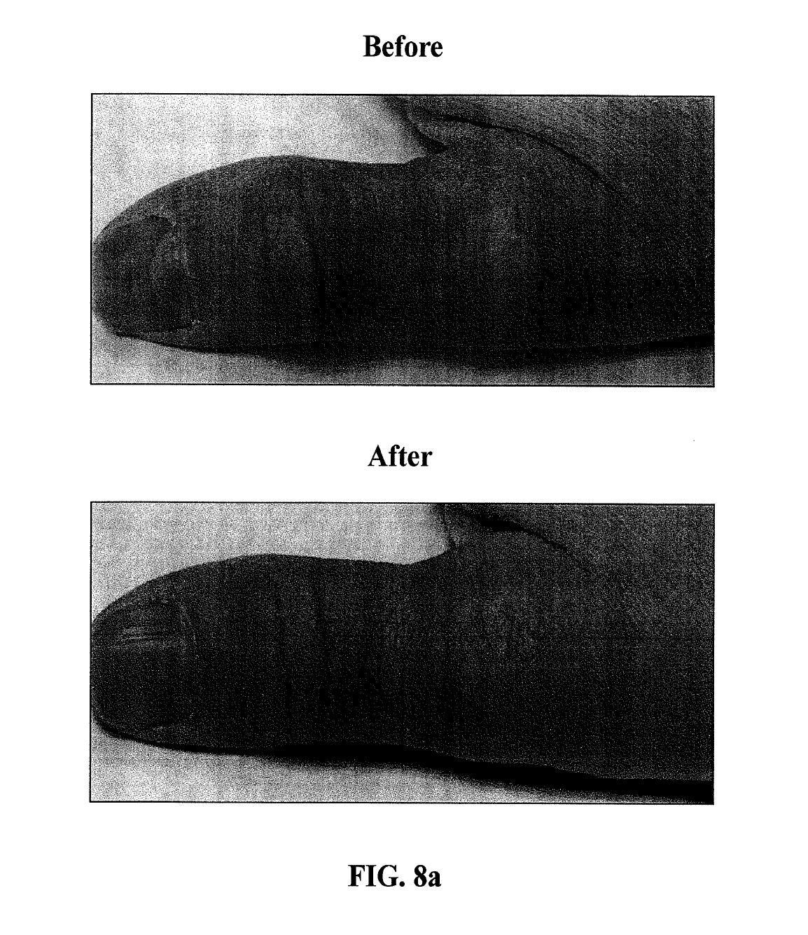

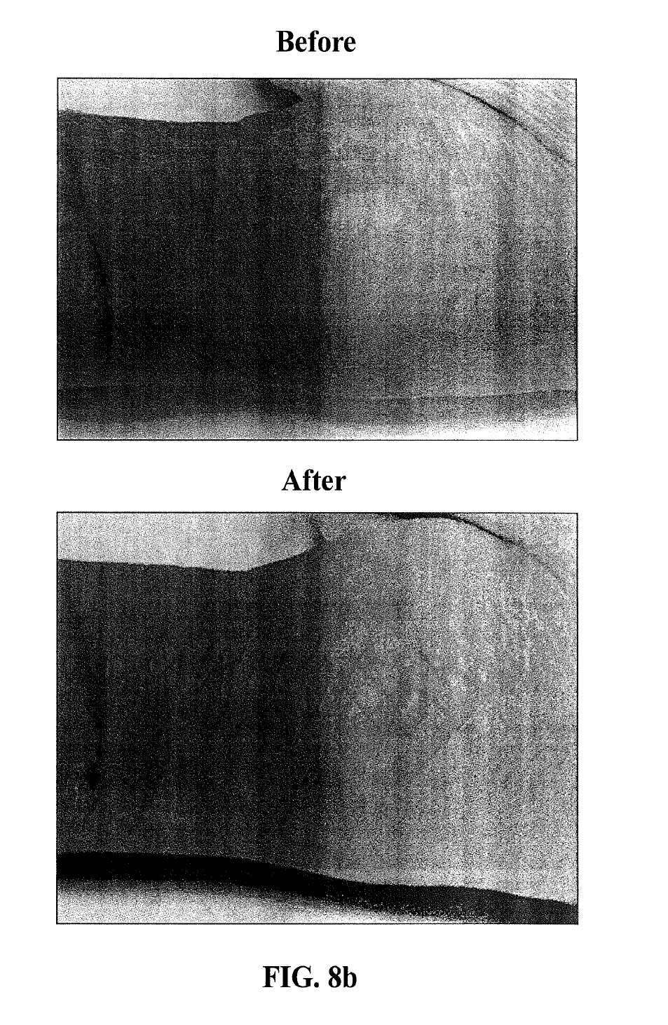

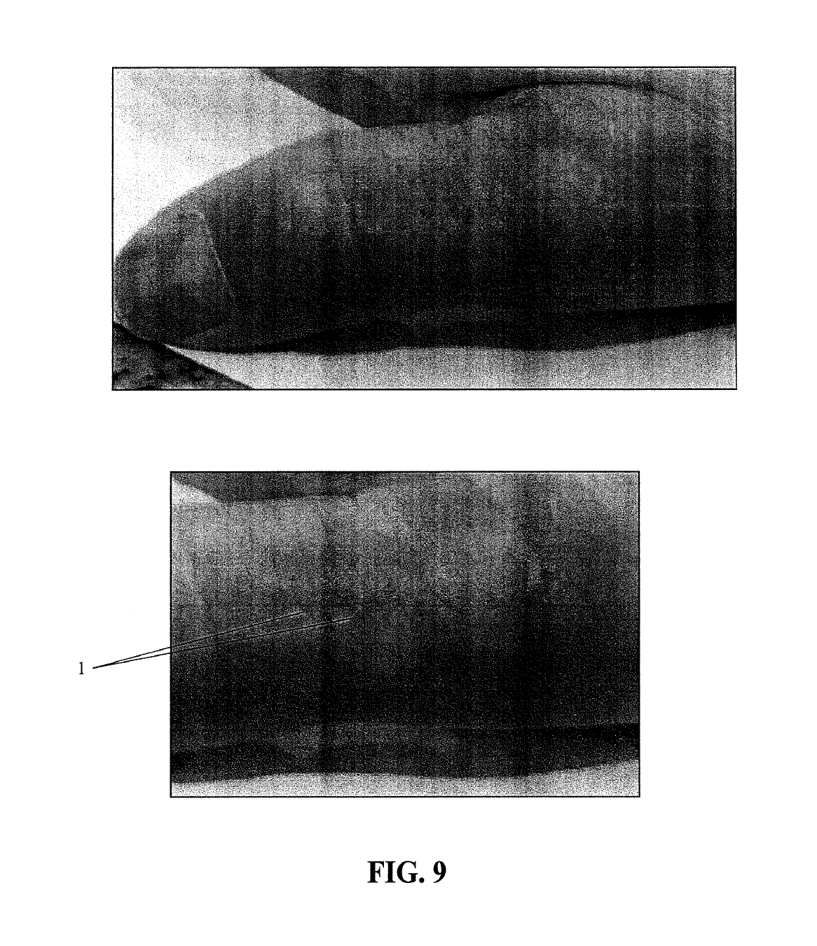

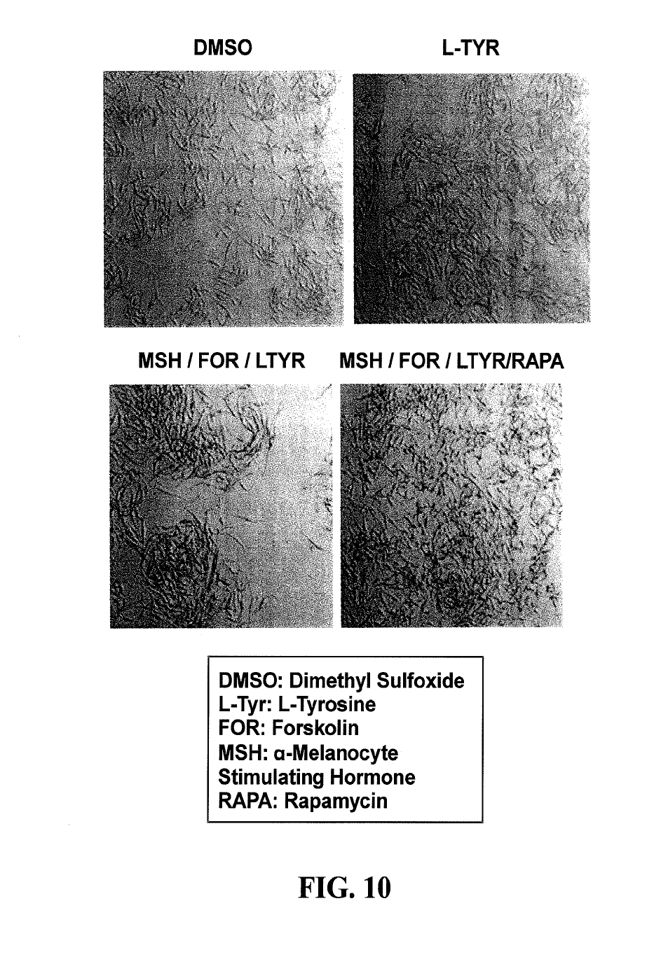

As depicted in FIG. 4, melanogenesis increases in human melanocytes after treatment with each of forskolin, Alpha-MSH, and L-dopa for 96 h. FIG. 5, combination of Forskolin (cAMP Activator) and Alpha MSH (Melanogenesis Activator) leads to enhanced melanogenesis in primary human melanocytes. In FIG. 5, Primary Human melanocytes were treated with each agent or the combination for 96 h. FIG. 6 depicts the increased melanogenesis caused by treatment of melanocytes with forskolin, L-dopa, and Alpha-MSH compared to untreated melanocytes. Additionally, FIGS. 7, 8, 9a and 9b depict the increased melanogenesis and proliferation of melanocytes in primary human melanocytes treated with a combination of forskolin and alpha-MSH compared to untreated melanocytes. FIG. 7 depicts a Western blot for protein expression of survival signals in primary human melanocytes treated with FGF, stem cell factor (SCF), forskolin and alpha-MSH, as compared to untreated melanocytes. FIGS. 8a and 8b depict the results of increased melanogenesis and proliferation of melanocytes three weeks after topical application twice a day to a subject. FIG. 9 depicts the results of increased melanogenesis and proliferation of melanocytes one week after commencement of treatment to a subject. Designation of 1 indicates the darkening (repigmentation) of skin surrounding hair follicles. FIG. 10 depicts increased melanogenesis in cultured primary melanocytes treated with L-Tyrosine, Alpha-MSH, Forskolin, Rapamycin or combinations thereof for 96 hours.

The treatment of vitiligo with a composition comprising rapamycin or a composition comprising rapamycin and FGF, or a composition comprising rapamycin, and at least one of antioxidants, melanogenesis substrates, melanogenesis stimulators and cAMP activators as other ingredients has many desired effects in management of skin and skin disorders, including anti-ageing, anti-wrinkle and/or an anti-cellulite effects, minimizing the appearance of wrinkles, blemishes, skin lines, acne, dry skin, xerosis, ichthyosis, dandruff, brownish spots, keratoses, melasma, lentigines, age spots, skin pigmentation, topical inflammation, liver spots, pigmented spots, wrinkles, blemishes, skin lines, oily skin, acne, warts, eczema, pruritic skin, psoriasis, inflammatory dermatoses, disturbed keratinization, bacterial infection, fungal infection, wound healing, and skin changes associated with aging, hypomelanotic disorders such as post inflammatory hyopmelanosis, infectious or parasitic hypomelanosis (e.g. Pityriasis (Tinea) Versicolor, Leprosy, Treponematoses, Onchocerciasis, Post-kala-azar Dermatosis, Herpes Zoster), Halo Nevus, Melanoma associated leukoderma, hypomelanosis from physical agents, hypomelanosis from chemical or pharmacological agents, hypomelanosis of ito, nevus depigmentosus, Hypopigmented Mycosis Fungoides, Scleroderma and Lichen Sclerosus associated hypomelanosis, Lupus Erythematosus associated hypomelanosis, sarcoidosis associated hypomelanosis and Pityriasis Alba. In particular, the rapamycin compositions treat skin pigmentation, topical inflammation and pigmented spots.

Cosmetically Acceptable Vehicle

In some embodiments, the present invention is directed to a composition comprising a cosmeceutically acceptable vehicle. As used herein, “cosmeceutically acceptable vehicle” refers to a component of the composition suitable for acting as a diluent, dispersant, or carrier for an active ingredient. In some embodiments, a cosmeceutically acceptable vehicle comprises materials commonly employed in skin care products such as water, liquid or solid emollients, silicone oils, emulsifiers, solvents, humectants, thickeners, powders, propellants and the like. Other agents which can be employed in the vehicle include fibroblast growth factor (FGF), stem cell factor, endothelins, other growth factors, tromethamine, glutathione peroxidase, catalase, vitamin E, sphingoid and phospholipid derivatives, antioxidants and vitamins, antiinflammatories, botanical agents, moisturizing agents, skin whitening agents, peptides, caffeine, sunscreens and UV absorbers, L-dopa, alpha-melanocyte stimulating hormone, cAMP activators, forskolin, colforsin, L-Tyrosine, N-Acetyl L-Tyrosine, other amino acids or combinations thereof. For example, in one embodiment, a cosmeceutically acceptable vehicle suitable for use in the present invention comprises water, glycerin, hydrogenated polyisobutene, cetearyl alcohol, ceteareth-20, macadamia integrifolia seed oil (macademia nut oil), dimethicone, tocopheryl acetate, stearoxytrimethylsilane, stearyl alcohol, panthenol, farnesol, benzyl alcohol, phenoxyethanol, acrylates/C10-30 alkyl acrylate crosspolymer, sodium hydroxide, citric acid. In another embodiment, a cosmeceutically acceptable vehicle comprises water, petrolatum, glyceryl polymethacrylate, dicaprylyl ether, glycerin, dimethicone, glyceryl stearate, cetyl alcohol, prunus amygdalus dulcis (sweet almond) oil, PEG-30 glyceryl stearate, tocopheryl acetate, benzyl alcohol, phenoxyethanol, sodium hydroxide, acrylates/C10-30 alkyl acrylate crosspolymer, disodium EDTA, propylene glycol. Another example of a cosmeceutically acceptable vehicle suitable for use with the present invention is Cetaphil®, which is commercially manufactured by Galderma Laboratories. In other embodiments, the cosmeceutically acceptable vehicle comprises additional agents including, but not limited to sphingoid and phospholipid derivatives (e.g., ceramides, phytosphingosine, sphingosine, pseudoceramides, phospholipids, lysophospholipids); antioxidants and vitamins (e.g., catalase, vitamin E, tocopherol and derivatives, ascorbic acid and derivatives, niacinamide and derivatives, vitamin complexes, alpha-lipoic acid, retinol and derivatives, panthenol); antiinflammatories (e.g., bisabolol, allantoin, phytantriol, Coenzyme Q10, Idebenone); botanical agents such as polyphenolics, flavonoids or isoflavones; moisturizing agents (e.g., amino acids, hyaluronic acid and derivatives, creatine and derivatives, trimethylglycine, myoinositol, pyroglutamatic acid and derivatives, taurine, guanidine and derivatives and hydroxy acids); skin whitening agents (e.g. kojic acid, arbutin, vitamin C and derivatives, hydroquinone); peptides, modified peptides, protein hydrolysates, or combinations thereof.

Formulation Table 1 below discloses an example composition of the present invention.

FORMULATION TABLE 1
Rapamycin (FW = 914.17)
200 mg in 500 ul DMSO (0.4 mg/ul)

| | Desired | | | | Amount | | |
|---|---|---|---|---|---|---|---|
| | Percent | Total | Amount | | Active | | |
| Desired | Conc. | Volume/Mass | Active | | Ingredient | | Amount of |
| Conc. | (% w/v) | Desired | Ingredient | | (mgs) | | Stock |
| 0.005 | 0.5 | 15 g | 0.075 | g | 75 | mg | 187.5 μl |
| | | | | | | | of the 0.4 |
| 0.001 | 0.1 | 15 g | 0.015 | g | 15 | mg | 37.5 μl |
| | | | | | | | of the 0.4 |
| 0.0005 | 0.05 | 15 g | 0.0075 | g | 7.5 | mg | 18.75 μl |
| | | | | | | | of the 0.4 |
| 0.0001 | 0.01 | 15 g | 0.0015 | g | 1.5 | mg | 3.75 μl |
| | | | | | | | of the 0.4 |
| 0.00005 | 0.005 | 15 g | 0.00075 | g | 0.75 | mg | 1.875 μl |
| | | | | | | | of the 0.4 |

In some embodiments, the present invention comprises a cosmeceutically effective amount of rapamycin. As used herein, the term “cosmeceutically acceptable amount” refers to an amount of rapamycin necessary to achieve a desired result. For example, in some subjects being treated, the cosmeceutically effective amount of rapamycin is dependent on the number of cells, or area of the skin to be treated. In some embodiments, a composition of the present invention comprises from about 1% to 0.00001% rapamycin, from about 0.5% to 0.1%, or from 1% to 0.1%. The Formulation Table 1 shows exemplary calculations for producing products of 0.01% and 0.005% rapamycin, similar calculations can be used to produce a cosmeceutically acceptable composition at the desired concentration. For example, in some embodiments, Formulation Table 1 discloses the amount of ingredients that may be used in any combination in the composition with 10 g of vehicle: addition of about 15 mg of rapamycin (about 0.15% by weight), addition of about 0.5 μg to about 1.5 μg of fibroblast growth factor (about 0.00005% to 0.00015% by weight), addition of about 0.5 g of tromethamine (about 0.5% by weight), addition of about 30 mg of glutathione peroxidase (about 0.3% by weight), addition of about 30 mg of catalase (about 0.3% by weight), and addition of about 500 mg of vitamin E (about 5% by weight). In another embodiment, the rapamycin and FGF together, comprise about 0.15% by weight in a cosmeceutically acceptable vehicle.

In some embodiments, the cosmeceutically acceptable vehicle will usually form from about 80% to about 99.999%, from about 95% to about 99.985% or at least about 99.985% by weight of the composition, and can, in the absence of other cosmetic adjuncts, form the balance of the composition. In one embodiment, an amount of rapamycin is maintained at a concentration of about 0.15% by weight in a cosmeceutically acceptable vehicle. In another embodiment, an amount of fibroblast growth factor (e.g. recombinant human basic fibroblast growth facter—FGF2) is maintained at a therapeutically effective concentration of about 0.0000001% to 0.1%, or about 0.000001% to 0.00001%, or about 0.00005% in a cosmeceutically acceptable medium. In another embodiment, an amount of forskolin or colforsin is maintained at a therapeutically effective concentration of about 2% to 0.001%, or about 0.5% to 0.05%, or about 0.1% in a cosmeceutically acceptable medium. In another embodiment, an amount of alpha melanocyte stimulating hormone is maintained at a therapeutically effective concentration of about 1% to 0.001%, or about 0.05% to 0.005%, or about 0.1% in a cosmeceutically acceptable medium. In another embodiment, an amount of L-Tyrosine (or N-Acetyl L-Tyrosine) is maintained at a therapeutically effective concentration of about 5% to 0.01%, or about 3% to 1%, or about 2% in a cosmeceutically acceptable medium. In another embodiment, an amount of L-DOPA is maintained at a therapeutically effective concentration of about 1% to 0.001%, or about 0.5% to 0.05%, or about 0.3% in a cosmeceutically acceptable medium. In another embodiment, an amount of catalase is maintained at a therapeutically effective concentration of about 0.1% to 0.00001%, or about 0.001% to 0.0001%, or about 0.0003% in a cosmeceutically acceptable medium. In another embodiment, an amount of vitamin E is maintained at a therapeutically effective concentration of about 10% to 1%, or about 7% to 3%, or about 5%.

In another embodiment, a therapeutically effective amount of about 0.15% to 2% rapamycin, about 0.1% to 2% forskolin/colforsin, about 1% to 5% L-Tyrosine/N-Acetyl L-Tyrosine, about 5% to 10% Vitamin E are maintained in a cosmeceutically acceptable medium

According to embodiments of the present invention, the composition can be formulated into a number of acceptable forms. For example, in some embodiments a skin care composition can be formulated as aqueous solution, a water-in-oil (w/o) emulsion, an oil-in-water (o/w) emulsion, a dispersion of lipids, an aqueous, water-alcohol, oil or oil-alcohol gel, a solid stick, a wet-wipe or an aerosol. In some embodiments, if the cosmeceutically acceptable vehicle itself is an (w/o) or (o/w) emulsion, it can contain from about 5 to about 50% of an oil phase and from about 47 to about 94.95% water, with respect to the weight of the whole composition.

Product Preparation, Form, Use and Packaging

To prepare the topical composition according to the present invention, the usual manner for preparing therapeutic and cosmetic skin care products may be employed. The active components are generally incorporated in a cosmeceutically acceptable carrier in a conventional manner. The active components can suitably be dissolved or dispersed in a portion of the water or another solvent or liquid to be incorporated in the composition.

In some embodiments, the composition may be in the form of conventional skin-care products such as a cream, gel, lotion or the like. In some embodiments, the compositions of the present invention can also be formulated as a so-called “rinse-off” product, e.g., a bath or shower gel, possibly containing a delivery system for the actives to promote adherence to the skin during rinsing. In other embodiments, the compositions of the present invention can be formulated as a “leave-on” product, i.e., a product to be applied to the skin without a deliberate rinsing step after its application to the skin.

The composition may be packaged in any suitable manner such as in a jar, a bottle, tube, roll-ball, or the like, in the conventional manner.

In some embodiments, the compositions described in the present invention may be applied one or more times daily to the portion of skin requiring treatment. In some embodiments, the present invention comprises topically applying a composition of the present invention one or more times daily for a period of about 2 to 12 weeks, and beyond. The product is intended to be used long-term. For example, FIGS. 8 and 9 depict topical treatment of a subject in need thereof with a composition of the present invention.

In one embodiment, a quantity of about 0.25 ml of a composition of the present invention is applied topically to the skin from a suitable container or applicator and spread over and/or rubbed into the skin using the hands or fingers or a suitable device. In some embodiments, if a composition is formulated as a “leave-on” product and does not require any gloves or special applicators for effective use.

In some embodiments, the present invention is directed to a method of treating vitiligo comprising the step of: topically administering to a subject in need thereof, a composition comprising a cosmeceutically acceptable vehicle and a cosmeceutically effective amount of rapamycin wherein the composition reduces the T-cell induced destruction of melanocytes. In other embodiments, the present invention is directed to a method of inhibiting T-cells from attacking melanocytes comprising the step of: administering to the melanocytes, from 0.1 μM to 100 μM rapamycin.

While not being bound to any particular theory, a composition comprising rapamycin reduces the T-cell induced destruction of melanocytes by inhibiting the clonal expansion of lymphocytes by reducing cell cycle progression from G1 to S phase in T-cells, and thus, T-cell proliferation in addition to increasing melanogenesis in melanocytes. Rapamycin (Sirolimus) is a natural product of the bacterium Streptomyces hygroscopicus that is approved by the FDA for: clinical use as an immunosuppressant. Rapamycin is currently recommended for use in conjunction with cyclosporine (and corticosteroids) to reduce or prevent graft rejection by the host. Rapamycin targets the serine-threonine kinase mammalian target of rapamycin (mTOR), which is bound to regulatory associated protein of mTOR (Raptor) and other proteins in the mTOR complex 1 (mTORC1). Rapamycin binds to cytosolic FK-binding protein 12 (FKBP12) in a manner similar to tacrolimus or pimecrolimus. Unlike these other drugs, which inhibit calcineurin (PP2B), the Rapamycin—FKBP12 complex inhibits mTORC1 and the mTOR signaling pathway. The immunosuppressive potency of rapamycin has generally been attributed to inhibition of the clonal expansion of lymphocytes by preventing cell cycle progression from G1 to S phase in T-cells and, thus, T-cell proliferation. More recent studies have shown that it also stimulated differentiation of T regulatory cells and modulates innate immune cell responses. In our studies, topical administration leads to a reversion of vitiligo phenotype and repigmentation of vitiligo affected skin. This is most likely due to its immunosuppressant effects but may also be associated with increased melanogenesis. Inhibition of phosphatidylinositol-3 kinase (PI3K—upstream in mTOR pathway) induces melanogenesis and melanocyte differentiation. Furthermore, inhibition of mTOR with rapamycin increases melanogenesis

### Example 1

As shown in FIG. 1, HL60 (myeloid derived) cells were treated with increasing doses of rapamycin (0.1-100 uM) and protein expression profile was analyzed by Western blot. The rapamycin treatment decreased expression of pS6K (phosphorylated ribosomal protein S6). The expression levels of Actin are maintained and serve as a loading control. Decreases in phosphorylated S6K indicate inhibition of mTOR in treated cells.

### Example 2

| | Amount per 10 gms | |
|---|---|---|
| Rapamycin | 15 | milligrams |
| Fibroblast Growth Factor 2 | 1.5 | micrograms |
| Tromethamine | 0.5% | |
| Glutathione Peroxidase | 1.3 | enzyme units |
| Catalase | 30 | milligrams |
| Vitamin E | 500 | milligrams |
| Vehicle (medium) | 10 | grams |

### Example 3

| Vitilgo Cream | Amount per 10 gms | |
|---|---|---|
| Rapamycin | 15 | mg (0.15%) |
| Basic Fibroblast Growth | 0.5 | μg (0.000005%) |
| Factor (FGF2) | | |
| Stem Cell Factor | 0.5 | μg (0.000005%) |
| Forskolin | 100 μL of 25 mM (0.1%) | |
| Alpha Melanocyte | 250 μL of 250 μM (0.01%) | |
| Stimulating Hormone | | |
| Glutathione Peroxidase | 1.3 | enzyme units |
| L-Tyrosine | 33 | mg (0.3%) |
| L-DOPA | 33 | mg |
| Catalase | 30 | μg (0.0003%) |
| Tromethamine | 0.5% | |
| Vitamin E | 500 | milligrams |
| Vehicle (medium) | 10 | grams |

### Example 4

| Vitilgo Cream | Amount per 10 gms | |
|---|---|---|
| Rapamycin | 15 | mg (0.15%) |
| Basic Fibroblast Growth | 0.5 | μg (0.000005%) |
| Factor (FGF2) | | |
| Forskolin/Colforsin | 100 μL of 25 mM (0.1%) | |
| Alpha Melanocyte | 250 μL of 250 μM (0.01%) | |
| Stimulating Hormone | | |
| L-Tyrosine | 200 | mg (2%) |
| L-DOPA | 33 | mg (0.3%) |
| Catalase | 30 | μg (0.0003%) |
| Tromethamine | 0.8% | |
| Vitamin E | 500 | milligrams (5%) |
| Vehicle (medium) | 10 | grams |

### Example 5

| Vitilgo Drug Cream | Amount in weight percent |
|---|---|
| Rapamycin | 0.15%-2% |
| Forskolin/Colforsin | 0.1%-2% |
| L-Tyrosine/N-Acetyl | 1%-5% |
| L-Tyrosine | |
| Vitamin E | 5%-10% |

Suitable dermatological medium

### Formulation Example

A 0.4 mg/μl Rapamycin composition is made in DMSO as a stock solution, from which an aliquot of 187.5 μls of the stock solution is added to 200 g of lotion or cosmeceutically acceptable vehicle and mixed thoroughly. An FGF composition is made following a similar protocol for a cosmeceutically acceptable vehicle and mixed thoroughly. Additional concentrations can be made from a concentrated stock solution by methods known to one of ordinary skill in the art. The cosmeceutical formulation (lotion) can be stored at ambient temperature for topical use on those areas of the skin wherein additional lipid production is desired.

While the present invention has been described with respect to what is presently considered to be the embodiments, it is to be understood that the invention is not limited to the disclosed embodiments. To the contrary, the invention is intended to cover various modifications and equivalent arrangements included within the spirit and scope of the appended claims. The scope of the following claims is to be accorded the broadest interpretation so as to encompass all such modifications and equivalent structures and functions.

All U.S. and foreign patent documents, all articles, brochures, and all other published documents discussed above are hereby incorporated by reference into the Detailed Description.

We claim:

1. A composition comprising: a) a cosmeceutically acceptable vehicle, b) rapamycin, c) a growth factor comprising fibroblast growth factor, stem cell factor or endothelins; and d) a cAMP activator.

2. A composition according to claim 1, wherein the composition comprises about 2 weight percent or less rapamycin.

3. A composition according to claim 1, wherein the composition comprises about 0.1 weight percent or less growth factor.

4. The composition according to claim 1, wherein the cAMP activator comprises forskolin or colforsin.

5. A composition according to claim 1, wherein the composition comprises about 2 weight percent or less cAMP activator.

6. The composition according to claim 1, further comprising a melanogenesis activator.

7. The composition according to claim 6, wherein the melanogenesis activator comprises: alpha melanocyte stimulating hormone, l-DOPA, L-Tyrosine (or N-acetyl L-Tyrosine), or L-phenylalanine.

8. The composition according to claim 6, wherein the composition comprises about 5 weight percent or less melanogenesis activator.

9. The composition according to claim 1, further comprising a melanogenesis substrate.

10. The composition according to claim 9, wherein the melanogenesis substrate comprises l-DOPA, L-Tyrosine (or N-acetyl L-Tyrosine), or L-phenylalanine.

11. The composition according to claim 9, wherein the composition comprises about 5 weight percent or less melanogenesis substrate.

12. The composition according to claim 1, further comprising an antioxidant.

13. The composition according to claim 12, wherein the antioxidant comprises vitamin E or catalase.

14. The composition according to claim 12, wherein the composition comprises about 10 weight percent or less antioxidant.

15. The composition according to claim 1, wherein the composition promotes the proliferation of melanocytes in a subject.

16. The composition according to claim 1, wherein the composition reduces the T-cell induced destruction of melanocytes.

17. The composition according to claim 1, wherein said composition is a topical composition.

18. The composition according to claim 1, wherein said composition is formulated as a leave-on product.

19. A composition comprising a) a cosmeceutically acceptable medium, b) a cosmeceutically effective amount of i) rapamycin, ii) growth factor, iii) cAMP activator, iv) melanogenesis activator or melanogenesis substrate, and v) antioxidants.

20. A method of altering skin pigmentation comprising the step of: topically administering to a subject a composition comprising rapamycin and a growth factor.

21. A method of treating vitiligo comprising the step of: topically administering to a subject in need thereof, a composition of claim 1, wherein the composition reduces the T-cell induced destruction of melanocytes.

22. A method of inhibiting T cells that target melanocytes comprising the step of: administering to the melanocytes, from 0.1 μM to 100 μM rapamycin.

23. A composition comprising: a) from about 0.15% to 2% rapamycin; b) from about 1% to 5% of a cAMP activator; c) from about 0.01% to 5% of a melanogenesis activator or melanogenesis substrate; and d) from about 5 to 10% of an antioxidant.

24. The composition of claim 23, wherein a cAMP activator is forskolin or colforsin.

25. The composition of claim 23, wherein a melanogenesis activator is alpha melanocyte stimulating hormone.

26. The composition of claim 23, wherein the antioxidant is glutathione peroxidase or vitamin E.

27. The composition of claim 23, wherein it is topically administered.

28. A method of treating vitiligo comprising the step of: topically administering the composition of claim 23 to a subject in need thereof.
